(19) United States
(12) Patent Application Publication
(10) Pub. No.: US 2005/0076461 A1
(43) Pub. Date: Apr. 14, 2005

(54) Retractable application cover

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/682,581

(22) Filed: Oct. 9, 2003

(51) Int. Cl. A61F 13/15

(52) U.S. Cl. 15/209.1; 15/184; 206/361; 604/1

(57) ABSTRACT

The retractable applicator cover comprises of an elongated tubular housing with two open ends each with a reduced inside diameter. The retractable applicator cover is placed over the applicator tip of the applicator to enclose the applicator tip. The retractable applicator cover may be slide longitudinally to expose the applicator tip for application. After application, the retractable applicator cover may be slide back to its original position to cover the used applicator tip to allow sanitary disposal of the applicator.

### BACKGROUND-FIELD OF INVENTION

The present invention relates generally to a retractable cover. More specifically, the present invention relates to a retractable cover for applicators such as swabs.

### BACKGROUND-DESCRIPTION OF RELATED ART

Applicator such as a swab has an applicator tip that may be made of an absorbent material such as cotton or foam. This applicator tip may be premoistened with a liquid or the applicator tip may be placed in contact with a liquid prior to application. In either case, the applicator tip needs to be protected from contaminants and to be kept sanitary prior to actual application.

Conventional methods of protecting or sealing the applicator tip include sealing the entire applicator in a plastic or aluminum film or placing the applicator in an indentation on a plastic sheet and sealing the top surface with a plastic film or aluminum foil. The plastic or aluminum seal must be ripped open to remove the applicator and exposed the applicator tip for application. Other sealing methods place the applicator between two sheets of paper and sealing the edge of the paper thereby sealing the applicator between the two sheets of paper. The paper will have to be torn open to remove the applicator and expose the applicator tip. Applicators may also be bulk packaged and sealed by placing multiple applicators in a container such as a paper or plastic box and sealing the box or wrapping a plastic film around the box to seal the box. The conventional packagings are not designed to allow the packaging to be reused to cover the applicator tip after use to allow sanitary disposal of the applicator.

None of the conventional packaging methods is easy to open, particularly, when the applicators are individually packaged. Also, none of the conventional packaging allows the applicator tip to be covered for disposal once the packaging is opened.

### SUMMARY OF THE INVENTION

The present invention is a retractable cover for applicators. In the preferred embodiment, the retractable applicator cover comprises of an elongated tubular housing with two open ends each with a reduced inside diameter approximately that of the outside diameter of the applicator handle. The retractable applicator cover is placed over the applicator tip of the applicator to enclose the applicator tip. The retractable applicator cover may be slide longitudinally to expose the applicator tip for application. After application, the retractable applicator cover may be slide back to its original position to cover the used applicator tip to allow sanitary disposal of the applicator.

Another embodiment of the retractable applicator cover comprises of an elongated tubular housing with an open end and a sealed end with an opening means provided between the sealed end and the open end. The open end has a reduced inside diameter approximately that of the outside diameter of the applicator handle. The retractable applicator cover is placed over the applicator tip of the applicator to enclose the applicator tip. After the sealed end is opened through the opening means, the retractable applicator cover may be slide longitudinally to expose the applicator tip for application. After application, the retractable applicator cover may be slide back to its original position to cover the used applicator tip to allow sanitary disposal of the applicator.

### BRIEF DESCRIPTION OF THE DRAWINGS

FIG. 1 shows a sectional view of the preferred embodiment of the retractable applicator cover.

FIG. 2 shows a sectional view of the preferred embodiment of the retractable applicator cover with the cover retracted.

FIG. 3 shows a sectional view of another embodiment of the retractable applicator cover.

FIG. 4 shows a sectional view of the embodiment of the retractable applicator cover shown in FIG. 3 with the sealed end opened.

FIG. 5 shows a sectional view of the embodiment of the retractable applicator cover shown in FIG. 3 with the cover retracted.

### DETAILED DESCRIPTION OF THE PREFERRED EMBODIMENT

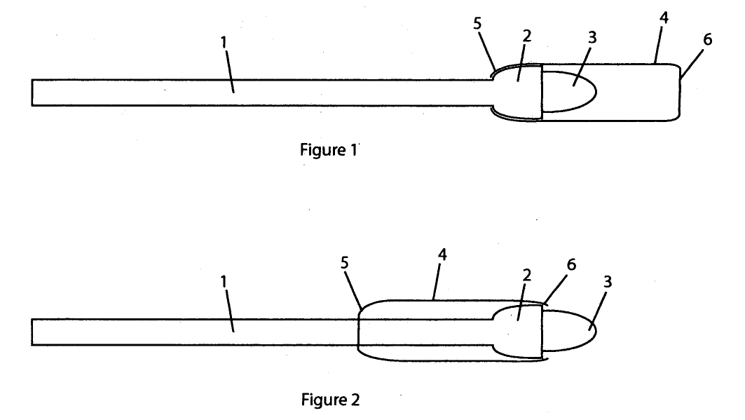

FIG. 1 shows the preferred embodiment of the retractable applicator cover. In the preferred embodiment, the retractable applicator cover comprises of an elongated tubular housing 4 with two open ends 5, 6 each with a reduced inside diameter approximately that of the outside diameter of the applicator handle 1. The retractable applicator cover is placed over the applicator tip 3 of the applicator to enclose the applicator tip 3 which is received in an enlarged cup 2 at an end of the applicator. The outside diameter of the enlarged cup 2 is approximately the same as the inside diameter of the retractable applicator cover. The retractable applicator cover may slide longitudinally along the applicator handle 1 to expose the applicator tip 3 for application as shown in FIG. 2. After application, the retractable applicator cover may slide back to its original position to cover the used applicator tip 3 to allow sanitary disposal of the applicator.

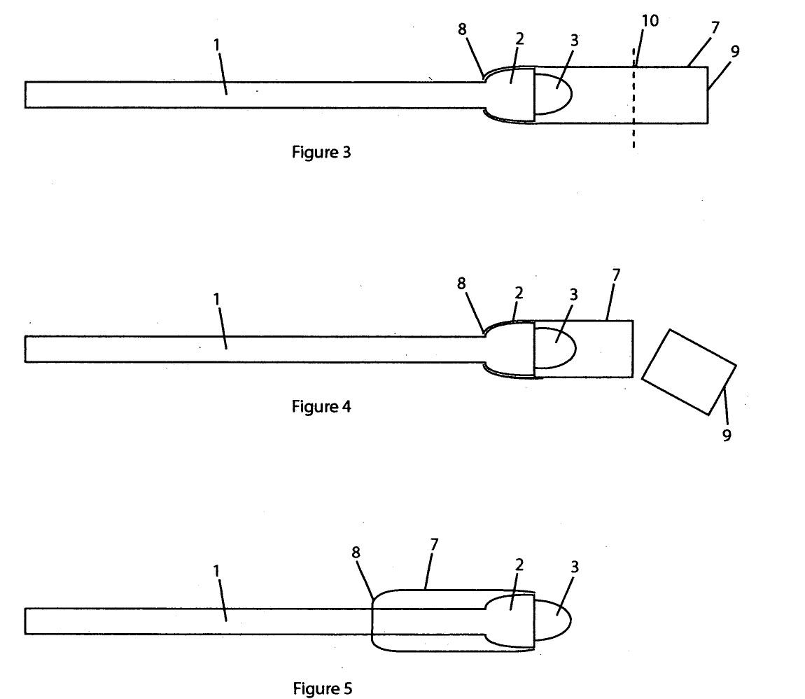

FIG. 3 shows another embodiment of the retractable applicator cover. In this embodiment, the retractable applicator cover comprises of an elongated tubular housing 7 with an open end 8 and a sealed end 9 with an opening means 10 provided between the sealed end 9 and the open end 10. The open end 10 has a reduced inside diameter approximately that of the outside diameter of the applicator handle 1. The retractable applicator cover is placed over the applicator tip 3 of the applicator to enclose the applicator tip 3 which is received in an enlarged cup 2 at an end of the applicator. The outside diameter of the enlarged cup 2 is approximately the same as the inside diameter of the retractable applicator cover. As shown in FIG. 4, after the sealed end 9 is opened through the opening means 10, the retractable applicator cover may slide longitudinally to expose the applicator tip 3 for application as depicted in FIG. 5. The opening means 10 may be in various forms such as a fracture line as shown in FIGS. 3 and 4 or a screw-on or snap-on cap. After application, the retractable applicator cover may slide back to its original position to cover the used applicator tip 3 to allow sanitary disposal of the applicator.

Although the description above contains many specificities, these should not be construed as limiting the scope of the invention but as merely providing illustrations of some of the presently preferred embodiments of this invention. Thus the scope of the invention should be determined by the appended claims and their legal equivalents, rather than by the examples given.

1. A retractable applicator cover comprising:
an applicator with an elongated handle and an applicator tip affixed to one end, and
an elongated tubular cover with two open ends covering the applicator tip of the applicator to enclose the applicator tip,
wherein the elongated tubular cover may slide longitudinally along the applicator handle to expose the applicator tip for application and, after application, the elongated tubular cover may slide back to its original position to cover the used applicator tip to allow sanitary disposal of the applicator.

2. A retractable applicator cover as in claim 1, wherein said applicator tip is received in an enlarged cup at the end of the applicator wherein the outside diameter of the enlarged cup is approximately the same as the inside diameter of the elongated tubular cover.

3. A retractable applicator cover as in claim 1, wherein each of said open ends of said elongated tubular cover has a reduced inside diameter approximately that of the outside diameter of the applicator handle.

4. A retractable applicator cover as in claim 2, wherein each of said open ends of said elongated tubular cover has a reduced inside diameter approximately that of the outside diameter of the applicator handle.

5. A retractable applicator cover comprising:
an applicator with an elongated handle and an applicator tip affixed to one end,
an elongated tubular cover with an open end and a sealed end covering the applicator tip of the applicator to enclose the applicator tip, and
an opening means provided between the sealed end and the open end of said elongated tubular cover,
wherein, after the sealed end is removed by the opening means, the elongated tubular cover may slide longitudinally along the applicator handle to expose the applicator tip for application and, after application, the elongated tubular cover may slide back to its original position to cover the used applicator tip to allow sanitary disposal of the applicator.

6. A retractable applicator cover as in claim 5, wherein said opening means is a fracture line.

7. A retractable applicator cover as in claim 5, wherein said opening means is a removable cap.

8. A retractable applicator cover as in claim 5, wherein said applicator tip is received in an enlarged cup at the end of the applicator wherein the outside diameter of the enlarged cup is approximately the same as the inside diameter of the elongated tubular cover.

9. A retractable applicator cover as in claim 5, wherein said open end of said elongated tubular cover has a reduced inside diameter approximately that of the outside diameter of the applicator handle.

10. A retractable applicator cover as in claim 8, wherein said open end of said elongated tubular cover has a reduced inside diameter approximately that of the outside diameter of the applicator handle.
